(19) United States
(12) Patent Application Publication
(10) Pub. No.: US 2019/0298530 A1
(43) Pub. Date: Oct. 3, 2019

(54) Radial Head Orthopedic Implant Apparatus and Method of Using Same

(71) Applicant: Ignite Orthopedics, Warsaw, IN (US)

(72) Inventors: Brian C. Hodorek, Winona Lake, IN (US); Matt J. Purdy, Winona Lake, IN (US); J. Michael Wiater, Beverly Hills, MI (US); Anand M. Murthi, Baltimore, MD (US); Matthew J. Smith, Columbia, MO (US); Derek J. Cuff, Venice, FL (US); Andrew Jawa, Cambridge, MA (US)

(73) Assignee: Ignite Orthopedics, Warsaw, IN (US)

(21) Appl. No.: 16/245,215

(22) Filed: Jan. 10, 2019

Related U.S. Application Data
(60) Provisional application No. 62/633,010, filed on Feb. 20, 2018.

(51) Int. Cl. A61F 2/38

(52) U.S. Cl. CPC A61F 2/3804; A61F 2002/30172

(57) ABSTRACT

An orthopedic implant apparatus, said apparatus comprising: a body, said body having a generally cylindrical shape, a flat bottom surface, and a top surface; at least one stem, said at least one stem connected to said bottom surface of said body; an articular component, said articular component having a bottom connecting side and a top bearing side; and a flexible component, said flexible component disposed between said body and said articular component, said flexible component attached to said top surface of said body and said bottom side of said articular component.

### BACKGROUND

### 1. Field of the Invention

The present invention relates generally to the field of orthopedic implants. More particularly, the present invention relates to orthopedic radial head implants.

### 2. Description of the Related Art

Trauma to the elbow joint frequently involves damage to the ligamentous support of the elbow and fractures of the osseous structures responsible for the skeletal integrity of the elbow joint. The proximal aspect of the radius, or radial head, is frequently injured either in isolation or in combination with injury to other bony or ligamentous structures of the elbow joint. The radial head may also be fractured in association with injuries to the forearm axis, including disruptions of the interosseous membrane between the radius and the ulna. Whether in isolation or in combination with other injuries, fractures of the radial head can be difficult to treat.

Fractures of the radial head are either reconstructable or unreconstructable. Despite various technical advances in the reconstruction of radial head fractures, a certain percentage of fractures are not amenable to reconstruction due to the degree comminution or severity of the fracture. In general, unreconstructable radial head fractures result from high energy trauma and are therefore frequently associated with significant injuries to other osseous or ligamentous structures of the elbow joint or forearm. In these cases, restoration of the stabilizing function of the radial head is critical to allow the ligaments of the elbow or forearm to heal in appropriate relationships, thereby restoring stability to the elbow or forearm. This stabilizing function depends, in part, upon re-establishing the appropriate distance between the capitellum and the proximal shaft of the radius.

The first widely used prosthetic radial head was introduced in the 1970's and was composed of silicone. Silicone implants placed in various joints throughout the body led to “silicone synovitis,” in which the silicone induces an inflammatory response within the joint. Further, silicone radial head prostheses were found to be incapable of resisting the stresses to which the radial head is subjected, rendering it less useful in stabilizing the injured elbow or forearm.

The difficulties apparent with silicone led to experimentation with metal radial head implants. These prostheses are fashioned from a single piece of metal (often titanium) and include a stem and a head portion. The head portion is shaped to approximate the anatomy of the radial head. These metallic prostheses are capable of resisting the compressive stresses to which the radial head is subjected, as has been demonstrated in several biomechanical studies. However, significant problems remain with these prostheses.

Anatomic and radiographic studies of the dimensions of the radial head reveal a disparity with currently available metallic prostheses. Therefore, it has been difficult to restore appropriate anatomic alignments within the elbow. Therefore, restoration of the appropriate relationship between the capitellum and proximal shaft of the radius has been very difficult to achieve with these prostheses. Additionally, the fact that these prostheses are fashioned from a single piece of metal has led to technical difficulties with insertion and removal. Surgeons have had difficulty with matching both the size of the stem to the canal of the proximal radius and the size of the head portion to the patient's native radial head. Removal of these non-modular components frequently requires release of the lateral ligaments of the elbow and the annular ligament, which binds the neck of the proximal radius to the proximal ulna. Thus, the elbow is frequently destabilized during removal of these prostheses.

Designers of prosthetic joint replacements in the hip, shoulder, knee and fingers have circumvented the above-mentioned difficulties by employing the use of modular components. Modularity allows for each aspect of a prostheses to be sized appropriately to its recipient anatomic site. The concept of modularity has only recently been applied to commercially available radial head prostheses. Currently available modular radial head prostheses employ a mechanism by which the head component is impacted over and onto the stem component. The surgical exposure must therefore allow sufficient room for the head to be maneuvered over the stem prior to being impacted. With impaction, the height of the prostheses is decreased, resulting in a shortening of the distance between the capitellum and the proximal shaft of the radius. Shortening this distance alters the bony anatomy such that the ligaments of the elbow joint are not held in their appropriate lengths and tensions. Instability of the elbow or inappropriate healing of the ligaments may result. Furthermore, removal of these prostheses is accomplished in the same manner as the above mentioned metallic implants, often requiring destabilization of the lateral aspect of the elbow joint.

Thus, a need exists for radial head implant devices that reap the benefits of modularity in radial head prosthetic replacement. Exemplary prior art devices, shown below, do not fully achieve this goal.

U.S. Pat. No. 9,763,792 discloses a system for replacing an end of a bone, such as a radial bone, with a prosthesis. In exemplary embodiments, the prosthesis is a radial head prosthesis having a stem portion and a head portion. The head portion may be configured to be (a) placed onto the stem portion by movement of the head and stem portions relative to one another transverse to a longitudinal axis of the stem portion, and then (b) rotated with respect to the stem portion to produce friction that firmly attaches the head portion to the stem portion.

U.S. Pat. No. 9,668,869 discloses an elbow prosthesis including a stem structure and an articulating component. The stem structure is operable to be positioned in a bone of a joint and includes a stem portion and a C-shaped body portion. The stem portion is operable to be positioned in the bone. The C-shaped body portion includes a first articulating surface bound by a medial wall and a lateral wall. The medial and lateral walls are separated by a first distance. The articulating component includes a second articulating surface positioned between a medial side surface and a lateral side surface. The medial and lateral side surfaces are separated by a second distance that is less than the first distance. The second articulating surface is configured to slidably communicate in a medial/lateral direction along the first articulating surface of the C-shaped body portion.

U.S. Pat. No. 9,655,726 discloses a radial-capitellar implant for surgical replacement of the capitellum of the humerus and, optionally, the head of the radius. The radial-capitellar implant includes a capitellar implant or surface replacement arthroplasty of the capitellum and a radial prosthesis for replacement of the head of the radius. In one embodiment the radial prosthesis includes an articular head which moveable articulates with a stem implantable in the radius.

U.S. Pat. No. 963,228 discloses a radial head implant including a head, a stem, and a locking mechanism, whereby the head laterally engages the stem, and an instrument for implanting and removing such implant is described.

U.S. Pat. No. 9,561,110 discloses an elbow prosthesis including a capitellar implant having an articulating head and a stem. The articulating head can have a first articulating surface positioned generally between a lateral side and a medial side. A passage can extend through the articulating head from the lateral side to the medial side. The articulating head can define a counterbore formed at the lateral side and that is concentric with the passage. According to other features, the elbow prosthesis can include a coronoid implant that has a body and a stem. The body can have a superior articulating surface that includes a central ridge and an anterior buttress. The central ridge can be configured to accommodate articulation with a trochlea in an implanted position.

U.S. Pat. No. 9,039,779 discloses an elbow prosthesis having a first stem component attached to one of a humerus and an ulna, a second stem component attached to the other of the humerus and the ulna, and a joint disposed between and coupling the first stem component and the second stem component to permit relative movement between the first stem component and the second stem component about a first axis. The elbow prosthesis may additionally include a condyle extending from the joint and including an axis of rotation that is eccentric from the first axis.

U.S. Pat. No. 9,034,050 discloses an elbow prosthesis including a capitellar implant that has an articulating head and a stem. The articulating head can have a first substantially hemispherical portion and a second portion that collectively extend between lateral and medial sides of the articulating head and are separated by a plane. The first and second portions can generally extend between the lateral and medial sides. The second portion can generally have an attachment lobe that extends on the lateral side and that defines a passage therethrough. The passage can extend at least partially on the second portion.

U.S. Pat. No. 8,920,509 discloses a prosthesis system for replacement of a head portion of a proximal radius can include an articulation component having a first connection portion. A first head component can have a second connection portion that connects to the first connection portion. A second head component can have a third connection portion that connects to the first connection portion. The second head component can have a distinct dimension from the first head component. A stem component can include a fourth connection portion that connects with either of the first or second head components. The stem component can have a stem anchoring portion that connects to the radius.

U.S. Pat. No. 8,535,382 discloses a prosthesis system for replacement of a head portion of a proximal radius. The system can include a first polymeric articulation component having a first locking portion and a metal head component having a second locking portion. The second locking portion can mate with the first locking portion to form a first locking mechanism to initially couple the first articulation component to the head component. The head component can define a locking channel. The system can also include a stem component having a protrusion receivable in the locking channel. The protrusion can define a bore, and the stem component can be adapted to be coupled to the radius. The system can also include a fastener received through the locking channel and into the bore to provide a second locking mechanism that couples the head component to the stem component.

U.S. Pat. No. 8,425,615 discloses a modular prosthesis system for replacement of a head portion of a radius. The prosthesis system includes a head component having a first connection portion that connects to a second connection portion and a collar component having the second connection portion and a third connection portion. The system also includes a stem component including a fourth connection portion that connects with the third connection portion, the stem component having a stem anchoring portion that connects to the radius. The collar component provides the modular geometry to the prosthesis without having to have an increased number of head components and stem components with variable lengths and angles.

U.S. Pat. No. 8,366,781 discloses a modular prosthesis system for replacement of a head portion of a proximal radius includes a monolithic stem component, a head component, and a locking mechanism formed by the stem and head components. The stem component defines a stem anchoring portion having a longitudinal axis and configured to couple to the proximal radius, and a dovetail-shaped first mounting portion on a first end face that extends in a first direction transverse to the longitudinal axis. The head component has a dovetail-shaped second mounting portion on a second end face opposite the first end face slidably engaged with the first mounting portion along the first direction. One of the first and second mounting portions intersects the longitudinal axis. The locking mechanism is formed at an interface between the stem and head components and is engaged through relative translational movement between the stem and head components. A related method is provided.

U.S. Pat. No. 8,100,980 discloses an artificial elbow joint including a humeral component made of metal and an ulnar component made of resins for replacing an elbow joint. The humeral component of this artificial elbow joint is configured by a substantially cylindrical trochlea and a stem extending from the trochlea that is inserted into the humeral; and the ulnar component is configured by a joint surface member which receives the trochlea of humeral component in a rotatable manner and a stem which extends from the joint surface member and is inserted into the ulna. The stem of the humeral component is curved gently downward overall so as to comply with the lordotic shape of the humeral, and the trochlea is turnable about the centerline of the stem.

U.S. Pat. No. 7,641,695 discloses a modular endoprosthetic radial head implant includes an end cap secured to a cannulated body held to bone by a fixation element. The fixation element supports the cannulated body on a resected radial bone end, for example. The fixation element of one embodiment features a threaded stem with a spherical head. The stem fits distally into the cannulated body and extends through a hole formed by the distal end of the cannulated body. The spherical head nests inside the cannulated body. Polyaxial alignment between the cannulated body and the fixation element is locked using a jam nut tightened inside the cannulated body. An end cap fits into place proximally on the cannulated body. The end cap is formed of a joint surface material to provide a bearing surface.

U.S. Pat. No. 7,637,908 discloses a system for intramedullary subchondral support fixation of radial head fractures is disclosed. An illustrative embodiment of the system includes an insertion plate having a head segment, a neck segment extending from the head segment, at least one shaft segment extending from the neck segment and at least one tail segment extending from the at least one shaft segment. A plurality of fastener openings may be provided in the insertion plate and a plurality of fasteners may extend through at least two of the plurality of fastener openings, respectively. A method for intramedullary subchondral support fixation of radial head fractures is also disclosed.

U.S. Pat. No. 7,160,329 discloses a radial-capitellar implant for surgical replacement of the capitellum of the humerus and, optionally, the head of the radius. The radial-capitellar implant includes a capitellar implant or surface replacement arthroplasty of the capitellum and a radial prosthesis for replacement of the head of the radius. In one embodiment the radial prosthesis includes an articular head which moveable articulates with a stem implantable in the radius.

U.S. Pat. No. 6,656,225 discloses a modular prostheses system for replacement of the radial head portion of the radius bone, and methods for its use are disclosed. The system comprises a stem component comprising an anchoring portion and a mounting portion, and a head component having an open channel wherein the open channel is configured to connect to the mounting portion along an assembly axis that is transverse to a longitudinal axis of the stem component.

What is needed in the art is a modular radial implant that provides the flexibility to prevent damage to the capitellum while maintaining the strength of the fully metal device.

### SUMMARY

In one exemplary embodiment, the present invention includes an orthopedic implant apparatus, said apparatus comprising: a body, said body having a generally cylindrical shape, a flat bottom surface, and a top surface; at least one stem, said at least one stem connected to said bottom surface of said body; an articular component, said articular component having a bottom connecting side and a top bearing side; and a flexible component, said flexible component disposed between said body and said articular component, said flexible component attached to said top surface of said body and said bottom side of said articular component.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

In the drawings:

FIG. 1 shows a side view of an apparatus in accordance with an alternative exemplary embodiment of the present invention.

FIG. 2 shows a perspective view of an apparatus in accordance with an alternative exemplary embodiment of the present invention.

Corresponding reference characters indicate corresponding parts throughout the several views. The exemplary embodiments set forth herein are not to be construed as limiting the scope of the invention in any manner.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail to avoid unnecessary obscuring of the present invention.

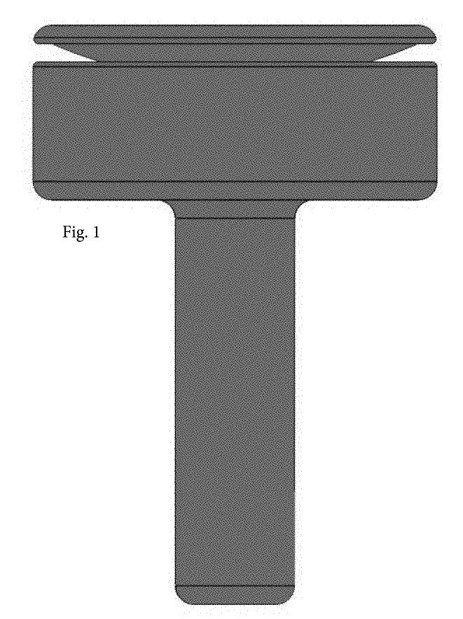

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

Furthermore, there is no intention to be bound by any expressed or implied theory presented in the preceding Technical Field, Background, Summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

While there are many acceptable exposure methods, the Kaplan interval in a line from the lateral epicondyle toward Lister's tubercle, with the forearm in neutral rotation, permits the collateral ligament to be left intact. In fracture dislocations, the exposure is through the traumatic opening in the ligament complex. Proximally, the ECRL origin is released with the anterior capsule to permit direct access to the front of the radial head.

Thus, the surgical technique for implanting a radial head device generally comprises templating the radial head prior to surgery to determine the appropriate level of resection; resecting the radial head with a surgical saw as close to the surgical neck as possible; preparing the canal for the stem using sequentially larger broaches; reaming the radial canal prepare the same for the implant; planning the neck of the radius to ensure a flat surface perpendicular to the canal for the head of the implant; inserting the trail implant into the radius to evaluate the stem size, head diameter and head height; and replacing the trial implant with the final implant.

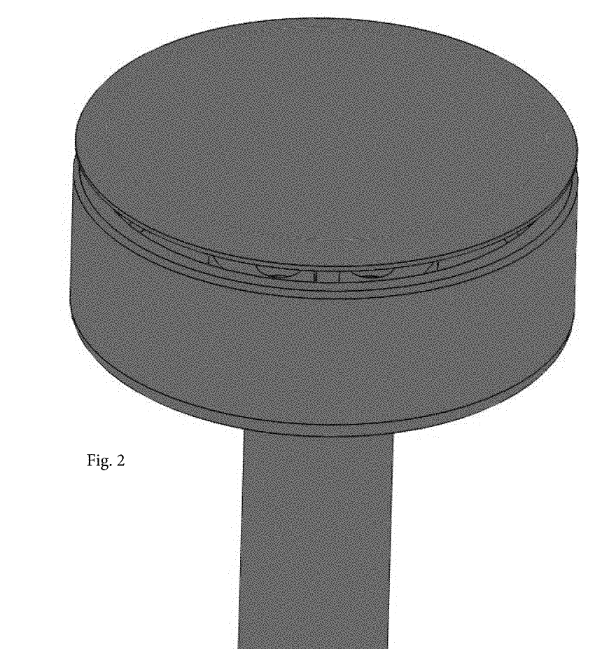

Referring generally to FIGS. 1-2, there is shown implant apparatus 1000. Apparatus 1000 generally comprises body 100 having distal stem 110 and proximal portion 120. Body 100 of apparatus 1000 comprises a biocompatible metal such as stainless steel, cobalt chrome, or titanium. Apparatus 1000 further comprises flexible component 130 attached to proximal portion 120 of body 100. Flexible component 130 may comprise silicone or any other flexible biocompatible material.

Referring still to FIGS. 1-2, apparatus 1000 further comprises “floating” articular component 140. Component 140 comprises a biocompatible metal such as stainless steel, cobalt chrome, or titanium. Component 140 further comprises concave upper surface 141. Surface 141 is a highly polished concave surface adapted to articulate against bone. As illustrated in FIGS. 1-2, component 140 further comprises lower surface 142 fixedly attached to flexible component 130.

Flexible component 130 can compress and/or tilt as it encounters on and off axis loading from articular component 140. Flexible component 130 functions as a “shock absorber” to protect the capitellum from the harsh solid metallic loading present in some prior art devices.

Referring still to FIGS. 1-2, body 100 of apparatus 1000 further comprises at least one stem 110. Stem 110 extends from the bottom of body 100 in a direction opposite flexible component 130. Stem 110 may comprise a smooth surface, a porous surface, or bone growth promoting material as desired. It is further contemplated that a plurality of stems 110 may be provided to attach body 100 to a human radius.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An orthopedic implant apparatus, said apparatus comprising:
a body, said body having a generally cylindrical shape, a flat bottom surface, and a top surface;
at least one stem, said at least one stem connected to said bottom surface of said body;
an articular component, said articular component having a bottom connecting side and a top bearing side; and
a flexible component, said flexible component disposed between said body and said articular component, said flexible component attached to said top surface of said body and said bottom side of said articular component.

2. The apparatus of claim 1, wherein said body is comprised of a biocompatible metal.

3. The apparatus of claim 1, wherein said biocompatible metal is selected from the group consisting of cobalt chrome, stainless steel, and titanium.

4. The apparatus of claim 1, wherein said plug is comprised of a biocompatible plastic.

5. The apparatus of claim 1, wherein said biocompatible plastic is selected from the group consisting of polyethylene, cross-linked polyethylene, and ultra-high molecular weight polyethylene.

6. The apparatus of claim 1, wherein said stem is porous.

7. The apparatus of claim 1, wherein said stem is smooth.
